(19) United States
(12) Patent Application Publication
(10) Pub. No.: US 2005/0260695 A1
(43) Pub. Date: Nov. 24, 2005

(54) Methods, compositions, devices, and kits for detecting mastitis

(75) Inventors: James E. Fleming, Spokane, WA (US); Jason Buck Somes, Spokane, WA (US); Stephanie Lynn Bernards, Spokane, WA (US); Tawyna May St. Pierre, Mead, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: GenPrime, Inc., Spokane, WA (US)

(21) Appl. No.: 10/948,076

(22) Filed: Sep. 23, 2004

Related U.S. Application Data
(60) Provisional application No. 60/505,297, filed on Sep. 23, 2003.

(51) Int. Cl. G01N 33/53; G01N 33/537; G01N 33/543; C12M 1/34

(52) U.S. Cl. 435/7.93; 435/287.2

(57) ABSTRACT

The present invention includes compositions, kits and methods useful for the detection of mastitis in an animal. These agents and methods are primarily directed to a method of detecting the presence of mastitis, including sub-clinical mastitis, in cows, involving incubating a sample of milk from the cow with an agent that binds to lactoferrin such as, e.g., a monoclonal antibody specific for lactoferrin, and then detecting bound lactoferrin. The invention includes lateral-flow immunoassay methods and devices for assessing the presence of lactoferrin in milk samples.

### BACKGROUND OF THE INVENTION

1. Field of the Invention

The present invention relates generally to methods, compositions and kits for the detection of mastitis in an animal. More specifically, the invention relates to devices and methods suitable for the detection of all types of mastitis, including sub-clinical mastitis, and which may be used in a variety of settings, including in the clinic, laboratory, or field.

2. Description of the Related Art

Bovine mastitis is an inflammation of the udder or mammary gland. It is a disease that is extremely costly to the dairy industry, with losses totaling 3 billion dollars annually as a result of infected cows. This loss results primarily from treatment of infected cows, discarded milk, death and premature culling, and reduced milk production (Gilson, W., 2001, Interpreting and using mastitis screen tests, The University of Georgia College of Agricultural and Environmental Sciences, Cooperative Extension Service, on-line publication). Of the four forms of mastitis, acute, chronic, clinical, and sub-clinical, the sub-clinical form accounts for most of the total cost associated with the disease. It has been estimated that over 1 billion dollars is lost in the U.S. because of reduced milk production attributable to sub-clinical mastitis.

The management of mastitis is critical to the economic success of the dairy farmer. Currently, mastitis is managed by maintaining hygienic housing, treating infected cows following early detection, and culling chronically infected cows. Detection of mastitis plays an important role in the overall management process since the condition needs to be treated quickly to minimize milk loss.

There are various detection methods employed by dairies to determine mastitis status, which include somatic cell counts (SCC), milk conductivity, standard microbiology assessment and the California Mastitis Test (CMT). Each of these methods is briefly discussed below.

The level of somatic cells, which are comprised of white blood cells, are indicative of infection since they reflect a response by the cow's immune system. They are an important part of mastitis management as the number of somatic cells often defines the severity of the infection (Harmon, R J., 2001, Somatic cell counts: a primer: Pp 3-9, Proc. Natl. Mastitis Count 40^{th} Annual Meeting, Feb. 11-14, 2001, Reno, Nev.). An uninfected cow usually has less than 150,000-200,000 somatic cells/ml. Somatic cell counts are routinely used because they correlate with infection status. However they characterize an advanced disease state, and are not helpful in predicting early onset of the disease. Moreover, somatic cell counts are usually performed in a laboratory and cannot be easily carried out in the field.

Electrical conductivity tests have been employed in limited conditions as a method for assessing sub-clinical mastitis but reports suggest that the predictive value of the method is poor (Seguya J M and Mansell P D, 2000, An evaluation of a hand-held electrical resistance meter for the diagnosis of bovine sub-clinical mastitis in late lactation under Australian conditions, Aus. Vet. J. 78:608-611; and Ruegg, P L, 2002, Milk quality and mastitis tests, online publication).

The microbiological examination of raw milk is a standard part of mastitis control. Milk samples are cultured and plated in a laboratory in order to identify and count microbes. This allows for the determination of the pathogens responsible for the infection. The complexity of the test and the need for formerly trained personnel indicate that this method is also ill-designed for use in the field (Houghtby G A, Maturin L J, and Koenig E K, 1993, Microbiologic). Bacteriology does not provide reliable test results, because up to 60% of mastitis milk samples do not contain viable bacteria.

The CMT is currently the only test routinely used in the field for assessing the severity of mastitis (Barnum D A and Newbould, F H S, 1961, The use of the California Mastitis Test for the detection of bovine mastitis, Canada Vet. J. 2:83-90; Ruegg P L, 2002, Milk quality and Mastitis tests, online publication; and Sargent J M, Leslie K E, and Shirley J E, 2001, Sensitivity and specificity of somatic cell count and California Mastitis test for identifying intramammary infection in early lactation, J. Dairy. Sci. 84:2018-2024). The test has been purportedly used to determine the presence of sub-clinical mastitis. It is inexpensive, easy to use and fast (tests take less than a minute). However, the test is highly subjective, does not detect all incidences of mastitis and is relatively insensitive. What the farmer needs is a rapid, simple, sensitive, inexpensive test for mastitis and sub-clinical mastitis that can be carried out in the field.

Clearly, there is a need in the art for methods and compositions for the rapid and sensitive detection of mastitis, including sub-clinical mastitis. The present invention meets these needs by providing an easy-to-use, sensitive, and accurate test for mastitis.

### BRIEF SUMMARY OF THE INVENTION

In one embodiment, the present invention provides a device for the detection of mastitis in an animal, comprising a carrier to which a milk sample obtained from an animal suspected of having mastitis can be applied; a labeled first binding agent that is mobile in the carrier when in the moist state; and a second binding agent that is immobilized in a first region of the carrier, wherein the binding agents bind lactoferrin.

In related embodiments of devices and kits of the invention, at least one of the binding agents is an antibody or fragment thereof that is specific for lactoferrin. In specific embodiments, the lactoferrin is cow, goat, sheep, human, or other mammalian lactoferrin. The antibodies may be monoclonal or polyclonal antibodies, or they may be fragments thereof, including, e.g., Fₐ, F_{b}, F_{c}, or Fₐ+F_{b} fragments.

In one embodiment of the device, the carrier comprises the following components: a liquid sample application wick; a conjugate pad comprising the labeled first binding agent; a base pad comprising the second binding agent; a sample pad comprising a blocking agent; and an absorbent pad. The device may further comprise a control binding agent that is immobilized in a second region of the carrier. The device may further comprise a detectable secondary agent that binds the labeled first binding agent.

In certain embodiments of the device, the labeled first binding agent is labeled with biotin, and the detectable secondary agent comprises strepavidin. The streptavidin may be conjugated to colloidal gold.

In another embodiment, the invention provides a kit for the detection of mastitis in an animal, comprising: a labeled first binding agent that specifically binds lactoferrin; and a device to which a liquid sample may be applied. In certain embodiments, the device is a carrier, a test strip, or a vessel.

In a related embodiment, the kit further comprises a second binding agent that specifically binds lactoferrin. The kit may further comprise a detectable secondary agent that binds the labeled first binding agent.

In specific embodiments of the kit, the labeled binding agents are antibodies. The antibodies may be, e.g., monoclonal antibodies, polyclonal antibodies, or fragments thereof.

In a related embodiment of the kit, the labeled binding agent is labeled with biotin. In another related embodiment, the secondary agent comprises streptavidin. In one embodiment, the streptavidin is conjugated to colloidal gold.

In yet another embodiment, the invention provides a method of detecting mastitis in an animal, comprising: incubating a sample of milk from an animal with a binding agent that binds lactoferrin; and detecting binding agent bound to the lactoferrin in the sample, thereby detecting mastitis in the animal.

In specific embodiments of the method, the labeled binding agents are antibodies. The antibodies may be, e.g., monoclonal antibodies, polyclonal antibodies, or fragments thereof.

In related embodiments of the method, the animal is a cow, a goat, a sheep, a horse, a pig, or a human, a camel, a llama, or any mammal.

In one embodiment of the method, the label is selected from the group consisting of: biotin, latex, colloidal gold, fluorescent dye, radiolabels, and an enzyme.

The invention provides, in yet another related embodiment, an analytical test device for detecting the presence of mastitis in an animal, comprising: a hollow casing having a liquid sample application region and a means permitting observation of a test result; and a test strip comprising a dry porous material contained within said hollow casing, said test strip communicating directly or indirectly with the exterior of said hollow casing through said liquid sample application region to receive applied liquid sample, said test strip having a capture line observable via said means permitting observation, said test strip, in the dry unused state, containing a labeled agent capable of specifically binding lactoferrin to form a first complex of said labeled agent and said lactoferrin which is indicative of bacteria, wherein said labeled agent is dry on said test strip prior to use and is released and mobile upon application of said applied liquid sample, and said test strip containing in said capture line a means for specifically binding said first complex, said means for binding being immobilized in said capture line; wherein said binding means binds said first complex to form a second complex, said second complex being observable via said means permitting observation, thereby to indicate the presence of mastitis in the animal.

In one embodiment of the device, the dry porous material is nitrocellulose, nylon, polyvinylidene fluoride, mixed cellulose esters, such as, e.g., nitrate and acetate, or polyethersulfone.

In certain embodiments, the device may further comprise a control line downstream from said capture line in said dry porous carrier to indicate that said liquid sample is conveyed beyond said capture line, and a control line observation aperture in said casing, said control line also being observable from outside said hollow casing through said control line observation aperture. In related embodiments, the control line contains a means for binding said labeled agent and wherein said means is immobilized in said control line. In specific embodiments, said means comprises an antibody or fragment thereof that specifically binds any portion of said labeled agent. The antibody may be a monoclonal antibody, a polyclonal antibody, or a fragment thereof, such as, e.g., F_{c}, Fₐ, F_{b}, and Fₐ+F_{b} regions of an antibody.

### BRIEF DESCRIPTION OF THE SEVERAL VIEWS OF THE DRAWING(S)

FIG. 1 is a graph depicting data from a capture ELISA using anti-lactoferrin antibodies against dilutions of raw and mastitis milk samples.

FIG. 2 is a graph depicting the difference in lactoferrin concentrations in mastitis and non-mastitis samples. Sample 1 is mastitis infection with Str. agalactiae, sample 2 is mastitis infection with S. aureus, and sample 3 is mastitis infection with Str. dysgalactiae. Samples 4, 5, and 6 are healthy non-mastitis milk. Sample 7 is a raw milk sample obtained from a dairy prior to pasteurization. Sample 8 is bovine lactoferrin at 1.25 ug/well, 0.625 ug/well, and 0.312 ug/well, respectively.

FIG. 3 is a diagram showing the basis of one embodiment of a capture antibody method used to determine the presence of lactoferrin in raw milk with monoclonal antibodies to bovine lactoferrin.

FIG. 4 is a diagram showing the layout of one embodiment of a capture antibody device used to determine the presence of lactoferrin in milk.

FIG. 5 is a photograph showing the major components of one embodiment of a lateral flow immunoassay cassette used for the detection of lactoferrin in milk.

FIG. 6 is a photograph demonstrating negative and positive test results. The round window to the left is the control window and the square window to the right is the test window. The presence of one line indicates a negative test (top). The presence of two lines indicates a positive test (bottom).

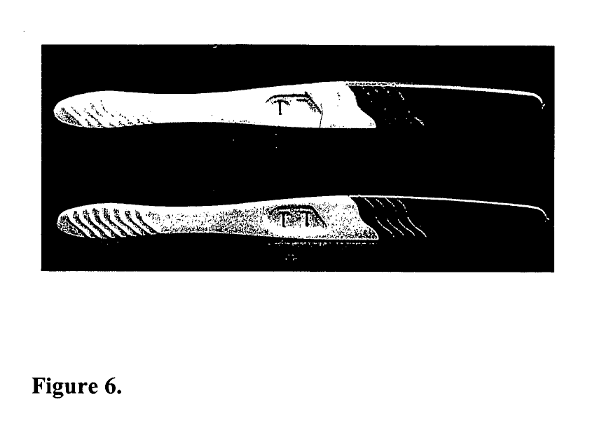

### DETAILED DESCRIPTION OF THE INVENTION

The present invention is directed to novel methods, devices and kits useful for the detection and diagnosis of mastitis. The invention is based, in part, upon the discovery that binding agents specific for mastitis-associated molecules, such as, e.g., lactoferrin, may be used to rapidly and sensitively detect the presence of such molecules in a milk sample, thereby detecting or diagnosing mastitis in the animal from which the milk was obtained. Although the skilled artisan would appreciate that the invention includes a variety of embodiments, one particular embodiment of the invention uses antibodies against lactoferrin to detect the presence of lactoferrin in milk samples. Furthermore, while the invention may be practiced by a variety of methods available in the art, in one embodiment, the invention is directed to methods and devices for lateral flow immunological detection of lactoferrin in a milk sample. Accordingly, the invention provides novel, rapid, and reliable methods and devices suitable for detecting and diagnosing mastitis in an animal by detecting the presence of a mastitis-associated molecule, such, e.g., lactoferrin, in a sample of milk obtained from the animal.

A. Methods of Detection

In one aspect, the invention provides novel methods of detecting the presence of mastitis in an animal by determining whether milk obtained from the animal contains a molecule or organism whose presence in the sample is indicative of the animal from which the sample was obtained having mastitis (e.g., a mastitis-associated molecule). In certain embodiments, the methods, apparatuses and kits of the invention are used to detect the presence of one or more specific forms of mastitis, which include acute, chronic, clinical, and sub-clinical. In one embodiment, the methods, apparatuses, and kits of the invention are used to detect sub-clinical mastitis. In certain embodiments, the invention includes methods of determining whether a threshold level of a mastitis-associated molecule is present in a sample. In other embodiments, the invention may be used to estimate or quantify the amount of a mastitis-associated molecule present in a sample and thus predict the level of infection. It is understood according to the invention that a mastitis-associated molecule may be any molecule or organism whose presence or absence, or increased or decreased levels, in a sample, is associated with the presence of any form of mastitis in the animal from which the sample was obtained. In addition, a mastitis-associated molecule may be any molecule having a detectable or measurable characteristic or property that is specifically associated with the presence of mastitis in the animal from which the sample was obtained. For example, a mastitis-associated molecule may have an altered conformation or may be associated with different molecules when present in a sample from an animal with mastitis as opposed to an animal without mastitis.

At a fundamental level, the invention involves combining a sample to be tested for the presence of a mastitis-associated molecule with an agent that binds a mastitis-associated molecule, and then determining whether any complexes containing both the binding agent and the mastitis-associated molecule are present. Accordingly, in one embodiment, the invention includes a method of detecting a mastitis-associated molecule in a sample, comprising incubating a sample with a binding agent that binds a mastitis-associated molecule and detecting the complex of binding agent bound to the mastitis-associated molecule, thereby detecting mastitis in the animal from which the sample was obtained. In one embodiment, methods of detection, e.g., ELISAs, are carried out in an indirect, direct, or capture formats. In one particular embodiment, the methods are carried out using sandwich capture with direct detection.

In one embodiment, the binding agent is labeled or comprises a label that is detectable either directly or indirectly. In certain embodiments, the binding agent is immobilized on a support, such as, e.g., a chromatography column or nitrocellulose, the sample is introduced to the immobilized binding agent, and the presence of a mastitis-associated molecule bound to the immobilized binding agent is determined, typically using a secondary binding agent that also binds to the mastitis-associated molecule. The immobilized binding agent is referred to as the capture binding agent, and the secondary binding agent, if it comprises a detectable label, is referred to as the labeled binding agent. It should be noted that, for all methods of the invention, the sample may be combined with the capture binding agent before, after, or simultaneously with the secondary or labeled binding reagent. Accordingly, in different embodiments, the capture binding agent may bind the mastitis-associated molecule alone or may bind the mastitis-associated molecule when it is bound to the secondary or labeled binding agent. In addition, either the capture binding agent or the second binding agent may bind another molecule that is complexed with or bound to the mastitis-associated molecule, e.g., wherein one of the binding agents is an antibody or fragment thereof specific for the mastitis-associated molecule and the second binding agent, either labeled or unlabeled, is an antibody or fragment thereof specific for a molecule bound to the mastitis-associated molecule.

The methods of the invention may be practiced using a variety of known techniques, depending, in part, upon the nature of the binding reagents. For example, the capture binding reagent may be immobilized in a column, and the sample may be passed through the column. In other embodiments, the invention may be practiced by various immunological assays, including immunosorbent assays such as, e.g., enzyme-linked immunosorbent assays (ELISAs) using antibodies specific for a mastitis-associated molecule. In one embodiment, the methods employ “sandwich” assays, wherein the mastitis-associated molecule is bound by a first binding agent and then detected by binding a second binding agent, which may be the same or different from the first binding agent. A wide variety of various binding assays are well-known in the art, and the skilled artisan would readily understand how to adapt such assays according to the present invention.

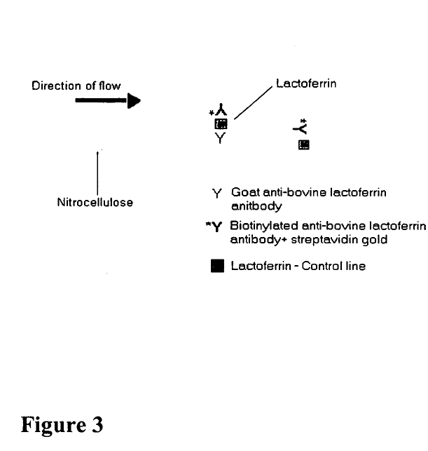

In one embodiment, methods of the invention are practiced using lateral flow techniques, including, e.g., lateral flow methods described in U.S. Pat. Nos. 5,622,871 and 6,352,862, and references and patents cited therein. According to one embodiment of the invention, methods using lateral flow techniques involve applying a liquid sample to one edge of a test strip comprising a porous material through which the sample can flow. A capture binding agent is immobilized in a region of the test strip (the capture line), such that when the sample flows over or through the capture line, mastitis-associated molecules present in the sample bind to the capture binding agent and are retained at the capture line. In certain embodiments, labeled binding agent is also present on the test strip, but it is mobile when wet by the liquid sample or bound to bacteria present in the liquid sample. Accordingly, as a liquid sample flows through the test strip, it comes into contact with both the capture binding agent and the labeled binding agent, resulting in the formation of a tertiary complex including labeled binding agent, mastitis-associated molecule, and capture binding agent at the capture line on the test strip. The presence of the complex is then determined via the label present in the labeled binding agent, either directly or indirectly using a secondary agent that interacts with the label. Typically, the signal generated by the label or secondary agent is visually detectable. A diagram of one specific embodiment of the method of the invention is provided in FIG. 3, and further details of specific embodiments of lateral flow methods and devices are provided infra.

1. Samples

Mastitis is an inflammatory condition that affects the mammary glands, and it is typically caused by infection with any of a variety of microorganisms. Mastitis affects a variety of animals, including, but not limited to, humans and other milk-producing animals, including dairy animals, such as cows, goats, and sheep. The invention may be used to detect the presence of mastitis in any susceptible animal from which a milk sample may be obtained. In certain embodiments, the invention is used to detect sub-clinical mastitis.

In one embodiment, the sample to be tested for the presence of a mastitis-associated molecule or organism in milk obtained directly from an animal suspected of having or being tested for mastitis. Milk may be obtained from the animal via any means available in the art, including manual and machine milking. The milk may be tested immediately or soon after being obtained, or it may be stored, typically under refrigeration, prior to testing.

2. Mastitis-Associated Molecules

The present invention may be used to detect or diagnose mastitis by detecting the presence of any of a variety of mastitis-associated molecules or organisms. In certain embodiments, the mastitis-associated molecule is indicative of sub-clinical mastitis. Other examples of mastitis-associated molecules include cytokines, immunogloblulins, triiodothynine (T₃), Interlukins, tumor necrosis factor-α (TNF-α), C-reactive protein, and nitric oxide metabolites. In addition, mastitis-associated molecules include molecules that bind or are are complexed with another mastitis-associated molecule, such as, e.g., iron and bacteria, which both bind lactoferrin.

In one embodiment, the invention is used to detect lactoferrin. As shown in the accompanying examples, the presence of lactoferrin in breast milk is an indicator of mastitis in the animal from which the milk is obtained. Lactoferrin is an iron-binding glycoprotein of the transferrin family, first isolated from milk but also found in most exocrine secretions as well as in the secondary granules of neutrophils. The many reports on its antimicrobial and antiinflammatory activity in vitro identify lactoferrin as important in host defense against infection and excessive inflammation. Most if not all lactoferrin actions are mediated through iron sequestration and/or interaction with a large variety of ligands including microbial cell wall components and cellular receptors, through its highly positively charged N-terminus. Lactoferrin exerts its effects on glandular epithelia, secretions, mucosal surfaces as well as in the interstitium and vascular compartments where it has been postulated to participate in iron metabolism, disease defense, and modulation of inflammatory and immune responses. Examples of specific lactoferrins that may be used according to the invention include lactoferrin-gamma, lactoferrin-alpha, lactoferrin-beta and lactoferrin residues.

In other embodiments, mastitis-associated molecules include other molecules associated with inflammation or infection, including for example, inflammatory mediators, and cytokines. Examples include, but are not limited to, Immunogloblulins, triiodothynine (T₃), Interlukins, tumor necrosis factor-α (TNF-α), C-reactive protein, and nitric oxide metabolites.

Examples of other bacteria that may cause mastitis and be mastitis-associated organisms include, but are not limited to, the gram-negative strains: Spirochaeta sp, Cristispira sp, Treponema sp, Borrelia sp, Leptospira sp, Campylobacter sp, Spirillium sp, Spirosoma sp, Pseudomonas sp, Xanthomonas sp, Phisobium sp, Methylococcus sp, Halobacterium sp, Acetobacter sp, Legionella sp, Neisseria sp, Moraxella sp, Flavobacterium sp, Brucella sp, Bordetrella sp, Francisella sp, Escherichia sp, Shigella sp, Salmonells sp, Citrobacter sp, Klebsiella sp, Enterobacter sp, Erwinia sp, Serratia sp, Hafnia sp, Edwardsiella sp, Proteus sp, Providencia sp, Morganella sp, Yersina sp, Vibrio sp, Pasterurella sp, Haemophilus sp, Desulfuromanas sp, Desulfovibrio sp, Desulfomanonas sp, Desulfococcus sp, Desulfobacter sp, Desulfobulbus sp, Desulfosarcina sp, Veillonella sp, Rickettsia sp, Rochalimeae sp, Coxiella sp, Ehrlichia sp, Cowdria sp, Wolbachia sp, Rickettsiella sp, Chlamydia sp, Mycoplasma sp, Ureaplasma sp, and Spiroplasma sp.

Examples of gram-positive bacteria that may cause mastitis include, but are not limited to: Micrococcus sp, Stomatococcus sp, Planococcus sp, Staphlycoccus sp, Deinococcus sp, Streptococcus sp, Sarcina sp, Pediococcus sp, Bacillus sp, Sporolactobacillus sp, Clostridium sp, Desulfotomaculum, Sporosarcina sp, Gardnerella sp, Streptobacillus sp, Lactobacillus sp, Listeria sp, Erysipelothrix sp, Corynebacterium sp, Mycobacterium sp, Nocardia sp, Haemophillus sp, and Heliobacter sp.

Typically, the invention contemplates the detection of a threshold level of a mastitis-associated molecule or organism in a sample. The skilled artisan would readily appreciate that the relevant threshold level depends, in large part, upon the sample being tested and the particular mastitis-associated molecule or organism being tested for. The determination of an appropriate threshold level for a particular sample to be tested may readily be determined by the skilled artisan based upon these and any other criteria established for a suitable application. Accordingly, the methods and devices of the invention may be optimized and/or the sensitivity adjusted such that a positive indication of the presence of a mastitis-associated molecule in a sample occur substantially only when the amount of mastitis-associated molecule is above a certain threshold level. The sensitivity of the methods and devices of the invention may be adjusted by a variety of means well understood in the art, including, for example, by varying the concentration of one or more of the following components of the system: capture binding agent, labeled binding, and detection agent.

In certain embodiments, positive indication of mastitis is made when the amount of mastitis-associated molecule in the sample is at least two times, at least three times, at least five times, at least ten times, or greater than ten times the amount in a control milk sample obtained from an animal known to not have mastitis. Lactoferrin concentration in normal milk is approximately 0.02% w/v, and milk from mastitic cows has an increased concentration of lactoferrin. In certain embodiments, a positive indication of mastitis is milk with a concentration of 0.5-0.10% w/v, 0.6% w/v, 0.7% w/v, 0.8% w/v, 0.9% w/v, or 0.10% w/v lactoferrin. In another embodiment, milk from a mastitic cow has a concentration equal or great than 0.10% w/v lactoferrin. According to Hagiwara et al., the range for healthy cows is 7-1150 μg/ml, and the range is 7-3600 μg/ml for mastitic cows (Hagiwara et al, Lactoferrin concentrations in milk from normal and subclinical mastitis cows, J Vet Med Sci, 2003. 65 (3). 319-23.) Accordingly, in certain embodiments, mastitis is indicated by a lactoferrin concentration of greater than 1000 μg/ml, greater than 1150 μg/ml, greater than 1500 μg/ml, greater than 2000 μg/ml, greater than 2500 μg/ml, greater than 3000 μg/ml, or greater than 3500 μg/ml. In one embodiment, a lateral flow device of the invention detects lactoferrin in the approximate range of 100 ng-500 μg/ml, 1 μg-500 μg/ml, 10-100 μg/ml, 10-500 μg/ml, 100-500 μg/ml, greater than 100 ng/ml, greater than 1 μg/ml, greater than 10 μg/ml, greater than 100 μg/ml, greater than 200 μg/ml, greater than 300 μg/ml, greater than 400 μg/ml, or greater than 500 μg/ml.

3. Binding Agents

The detection system of the invention is based, in large part, on the ability of an agent to bind a mastitis-associated molecule. In certain embodiments, the invention uses two binding agents, a labeled first binding agent and a second binding agent, i.e., capture binding agent, both of which bind to a mastitis-associated molecule, resulting in the formation of a ternary complex comprising capture binding agent, mastitis-associated molecule, and labeled binding agent. In one embodiment, the capture binding agent is used to immobilize the mastitis-associated molecule at a particular location, e.g., detection line or detection zone, where its presence may be determined. Typically, the labeled first binding agent binds to the mastitis-associated molecule to facilitate detection at the detection line or zone. It is understood, however, that the second or capture binding agent may be labeled. In one embodiment, if the second or capture binding agent is labeled, the label will be different than the label of the labeled first binding agent. In one example, one or both of the binding agents contain labels suitable for fluorescence resonance energy transfer (FRET) detection of a complex containing both binding agents. FRET labels and methods are widely available and known in the art.

In certain embodiments, the capture binding agent may be labeled such that a signal is detectable upon binding of the mastitis-associated molecule to the capture binding agent. Accordingly, labeled binding agents include binding agents that undergo a change upon binding, such that the agent emits a detectable signal. Additionally, the invention contemplates the use of biosensors, such as those described, e.g., in U.S. Pat. Nos. 6,540,890, 6,503,381, and 6,547,954 and references described therein.

Any of a variety of agents may be used, including, for example, polypeptides, sugars, and nucleic acids. The capture binding agent and the labeled binding agent may recognize the same or, preferably, different epitopes on the mastitis-associated molecule. In addition, either or both of the capture binding agent and labeled binding agent may bind molecules complexed with or associated with mastitis-associated molecule. Further, the capture binding agent may specifically recognize the labeled binding agent that binds a mastitis-associated molecule. In one embodiment, the capture binding agent specifically binds the labeled binding agent only when the labeled binding agent is bound to the mastitis-associated molecule.

In certain embodiments, the capture binding agent is an antibody specific for a mastitis-associated molecule, such as, e.g., lactoferrin. In other embodiments, the antibody is a monoclonal antibody, a polyclonal antibody, or a fragment thereof. Antibody fragments include all capable of binding to a target molecule, including, e.g., F_{c}, Fₐ, F_{b}, and Fₐ+F_{b} regions of an antibody. Furthermore, the capture binding agent and secondary or labeled binding agent may comprise the same binding moiety, although the labeled binding agent will further include a label. Antibodies specific to any mastitis-associated molecule, including lactoferrin, may be produced using methods widely known and available in the art. In addition, a variety of useful antibodies are commercially available. Antibodies specific for lactoferrin are commercially available RDI (Flanders, N.J.), Bethyl Laboratories (Montgomery Tex.), and Sigma-Aldrich (St. Louis, Mo.).

As used herein, an antibody or binding agent is said to be “immunospecific” or to “specifically bind” lactoferrin or another polypeptide if it reacts at a detectable level with a polypeptide, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

4. Labels

According to the invention, detection of a mastitis-associated molecule in a sample is accomplished through the use of a labeled binding agent. The labeled binding agent is an agent that binds specifically to mastitis-associated molecule and comprises a label. The label may be detected directly or indirectly, through the use of a secondary agent. The presence of the label may be detected by a variety of different methods, depending upon the nature of the label used. Accordingly, in certain preferred embodiments, the label may be detected visually. Examples of labels include, but are not limited to, biotin, latex, colloidal gold, fluorescent dyes and enzymes.

In certain embodiments, the labeled binding agent or secondary agent comprises a particulate label. A variety of such “direct labels” are known in the art, including, e.g., colored latex particles, gold sols, non-metallic colloids, and dye sols. Such labels can be used to produce an instant analytical result without the need for additional reagents to develop a detectable signal. They are robust and stable and can, therefore, be used readily in an analytical device that is stored in the dry state. Their release upon contact with a liquid sample can be modulated, for example, by the use of soluble glazes. In one embodiment, a particulate label is a latex label, such as a colored latex label that can be readily visible to the eye if it becomes bound at the detection zone.

In certain embodiments, a label may be a fluorescent compound, which can respond to applied electromagnetic energy, such as ultraviolet or visible light, to provide an emitted signal that can be detected visually or detected instrumentally. Labels include those used in fluorescence resonance energy transfer (FRET)-based detection methods, such as, e.g., fluorescein and rhodamine.

In certain embodiments, “indirect labels” may be used according to the invention. Such labels usually require the addition of one or more secondary or developing agents such as substrates before a visible signal is detectable. These agents include, amongst others, enzymes such as horseradish peroxidase and alkaline phosphatase.

In one embodiment, the label is horseradish peroxidase, and the secondary agent is ABTS, which reacts with horseradish peroxidase to produce a colored reaction that may be detected visually or by measuring absorbance using an appropriate filter, typically at 405 nm.

In another embodiment, the label is biotin, and the secondary agent comprises streptavidin. In one embodiment, the secondary agent is a streptavidin-gold conjugate.

Coupling of a label to a specific binding agent to produce a labeled binding agent may be performed by a variety of methods known in the art, including covalent bonding or by hydrophobic bonding. In one embodiment, an antibody may be labeled, e.g., using the BiotinTag Micro-biotinylation Kit from Sigma Chemical Co, St. Louis, Mo.

In embodiments wherein the invention is used to identify the presence of more than one analyte in a sample, the several different labeled binding agents may be used, each carrying a different label.

B. Detection Devices

The invention further provides apparatuses, devices and kits that may be employed according to methods of the invention to detect the presence of a mastitis-associated molecule in a sample and, thus, mastitis in the animal from which the sample was obtained. A variety of related devices have been described generally, particularly for methods of detecting pregnancy, and are described, e.g., in U.S. Pat. Nos. 5,622,871 and 6,352,862, and references and patents cited therein; Jones, K. D. (1999) Troubleshooting Protein Binding in Nitrocellulose Membranes (Part I)—Principles, IVD Technology; Chandler, J. et al. (2000) The Place of Gold in Rapid Tests, IVD Technology; Weiss, A. (1999) Concurrent Engineering for Lateral-flow Diagnostics, IVD Technology; and Paek, S. et al. (2000) Development of Rapid One-step Immunochromatographic Assay, Immun. Methods, 22:53-60. The devices of the invention and kits comprising the same may be readily prepared using known methods, including those described in the aforementioned references. In certain embodiments, these devices are prepared so that they may be stored in a dry form to facilitate stability and increase shelf-life.

In one embodiment, the invention provides a device or kit for the detection of a mastitis-associated molecule, comprising a material on which a capture binding agent is immobilized and a labeled binding agent.

In one embodiment, the invention includes a device or kit for the lateral flow detection of a mastitis-associated molecule, comprising an absorbant material that permits an applied sample, or mastitis-associated molecule therein, added to a first region of the absorbant material to move or flow to a second region of the absorbant material. The second region of the absorbant material comprises an immobilized capture binding agent, which specifically binds to a mastitis-associated molecule, thereby immobilizing the mastitis-associated molecule and facilitating its detection at the second region.

While the components of devices and kits of the invention will necessarily vary depending upon the particular method of detection being used, such devices and kits may include a carrier, which is also referred to in certain embodiments as a test strip. In one embodiment, the test strip contains a capture line (i.e. detection zone), a capture binding agent, and a labeled binding agent. Alternatively, the test strip may comprise only the capture binding agent or the labeled binding agent, and the other agent is supplied separately. In another embodiment, the test strip includes neither the capture binding agent or the labeled binding agent, and both binding agents are supplied separately. Typically, the capture binding agent (which, in certain embodiments, is an unlabeled binding agent) will be present on the test strip. In certain embodiments, the labeled binding agent may also be present on the test strip, or it may be separate from the test strip.

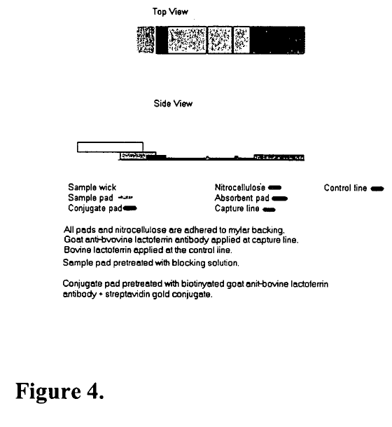

The test strip refers generally to the physical medium upon which the methods of the invention are practiced. The test strip is preferably a porous carrier material in the form of a strip or sheet to which during manufacture of the device, one or more reagents or physical components can be applied in spatially distinct zones. The test strip may comprise a single physical component, but usually the test strip will comprise multiple different physical components, including any of, e.g., a sample wick, a sample pad, a conjugate pad, a capture line, a control line, and an absorbent pad. The test strip may also be referred to as a cassette. Each of these components may be combined with any other individual or group of components. In one embodiment, the test strip is nitrocellulose, which permits the immobilization of proteinaceous reagents in a capture line without prior chemical treatment. If the test strip comprises paper, for example, the immobilization of the capture binding agent may be performed by chemical coupling using, for example, CNBr, carbonyldiimidizole, or tresyl chloride. Typically, and particularly where multiple components are used, each component will be adhered to a physical support, such as, e.g., mylar, plastic, or glass. One specific embodiment of a test strip is provided in FIG. 4.

In one embodiment, the test strip comprises a dry, porous carrier to which a liquid sample can be applied directly or indirectly. The dry, porous carrier may comprise a chromatographic strip, such as a strip of nitrocellulose, which may be advantageous as proteins are capable of directly binding to nitrocellulose. Nitrocellulose is available in a variety of pore sizes, thus facilitating the selection of a test strip suitable for any particular flow requirement with minimal effort. In certain embodiments, the nitrocellulose has a pore size of at least 1 micron, at least 5 microns, or 8-12 microns. Nitrocellulose sheets are available from Schleicher and Schuell GmbH. In one embodiment, a nitrocellulose paper provides a flow speed of 135 mm/4 min.

In certain embodiments, the test strip, e.g., nitrocellulose, may be backed to increase handling strength, e.g., with a moisture impermeable material, such as mylar, a polyester sheet, plastic, or glass.

In certain embodiment, a sample is applied to the test strip via the use of a sample wick, an optional component of the test strip. The sample wick can be made from any bibulous, porous, or fibrous material capable of absorbing liquid. The porosity of the material may be unidirectional or multidirectional. Porous plastics may be used, such as, e.g., polypropylene, polyethylene, and polyvinylidene fluoride. The sample sick may also be made from paper or other cellulosic materials, such as nitrocellulose.

In certain embodiments, the test strip may comprise an optional sample pad that comprises a blocking solution, which will interact with the sample prior to the sample contacting the labeled binding agent or capture binding agent to reduce non-specific binding and false positives. The sample pad is typically in direct moisture-conducting contact with the sample wick. Blocking solutions may be selected based upon the binding agent being used and are widely known in the art. For example, one blocking solution that may be used where the binding agents are antibodies is comprises bovine serum albumin (BSA). Other examples of blocking agents include, but are not limited to, casein, Blotto, trademarked blocking agents, chicken serum, BSA, fish gelatin, albumin, gelatin, Tween 20, Triton 100, glycerin, polyvinyl alcohol (PVA), polyvinyl pyrrolidone (PVP), polyethylene glycol (PEG), sodium dodecylsulfate (SDS), and sodium dodecylebenzene sulfonate (SDBS).

In one embodiment, the test strip comprises a conjugate pad, which comprises the labeled binding agent, and may further comprise a secondary reagent used to detect the presence of the labeled binding reagent. In one embodiment, the conjugate pad is a macroporous body wherein the applied liquid sample encounters the labeled binding agent. The use of a macroporous body is believed to facilitate the ease with which the labeled binding agent binds bacteria within the sample, as compared to the situation where the labeled binding agent is incorporated directly onto the dry, porous carrier. In certain embodiments, to facilitate migration of the labeled binding agent, the conjugate pad has a pore size at least ten times greater than the size of the labeled binding agent. In one embodiment, the conjugate pad comprises plastics material having an average pore size of e.g., at least 10 microns or at least 100 microns. The conjugate pad is preferably non-protein binding or readily blockable.

In certain embodiments, the conjugate pad is in direct moisture-conductive contact with the sample wick or the sample pad, and the detection zone on the test strip is spaced away from, typically downstream of, the region of contact between the test strip and the conjugate pad, as illustrated in FIG. 4.

The test strips further comprise a capture line (also referred to as a detection zone), which comprises the capture binding agent. The capture binding agent is immobilized at the capture line, thus facilitating the formation of a complex containing bound bacteria and labeled binding agent at the capture line, where it can be detected. In one embodiment, the capture binding agent is immobilized to the capture line of the test strip (e.g., nitrocellulose) via UV cross-linking. In other embodiments, the capture agent is immobilized at the capture line using any technique available in the art and suitable for the particular material being used, including, for example, hydrophobic interactions for polyvinylidene flouride (PVFD), the use of mixed cellulose esters (e.g., nitrate and acetate), the use of nylon (including, e.g., charge-modified or electrostatic(ionic) materials), electrostatic interactions using nitrocellulose or cellulose, and hydrophobic interactions using polyethersulfone.

In one embodiment, the capture line comprises a secondary agent, which facilitates detection of the labeled binding reagent.

The devices may optionally further comprise a control line comprising a control agent capable of binding the labeled binding agent. In one embodiment, wherein the labeled binding agent is an antibody, the control agent is an antibody directed against immunoglobulins. For example, where the labeled binding agent is a mouse monoclonal antibody, the control binding agent may be goat anti-mouse IgG1. Typically, the control line will be downstream of the capture line.

The device may also optionally comprise an absorbent pad, which is downstream from the capture line and optional control line.

The various components of the test strip are arranged, in one embodiment, as shown in FIG. 4. The spatial separation between the conjugate pad and capture line, and the flow rate characteristics of the porous materials of the test strip, can be selected to allow adequate reaction times during which the necessary specific binding can occur. Further control of these parameters may be accomplished by the addition of viscosity modifiers, e.g., sugars and modified cellulose, to the liquid sample to slow down migration.

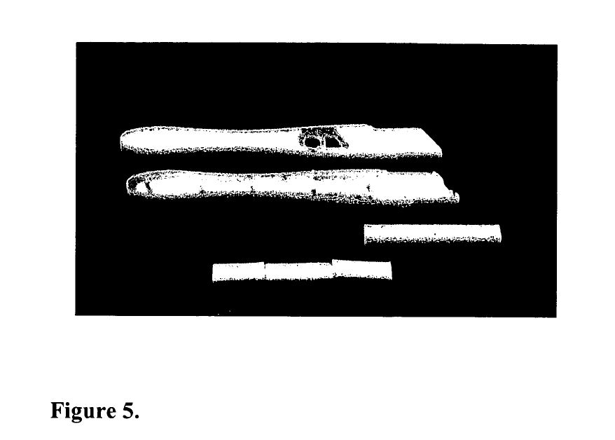

In certain embodiments, the test strips are contained within a moisture-impermeable casing or housing and the sample wick extends out of the housing and acts as a means for permitting the liquid sample to enter the housing. In another related embodiment, a sample may be applied to the test strip, e.g., to a sample wick or sample pad through an aperture in the housing. The housing is provided with means, e.g., appropriately placed apertures, which enable the capture line, and optional control line, to be observable from outside the housing so that the result of the detection assay can be observed. The housing may be provided with a removable cap that can protect a protruding sample wick during storage and can be placed over the sample wick while the assay is being performed. One embodiment of a device of the invention is shown in FIG. 5.

The invention further provides kits comprising labeled binding agent and capture binding agent. In certain embodiments, a kit comprises a test strip of the invention containing these binding agents. Kits may further comprise sample dilution buffers, blocking buffers, and/or instructions for use.

### EXAMPLES

### Example 1

### ELISA Detection of Mastitis Using an Anti-Lactoferrin Antibody

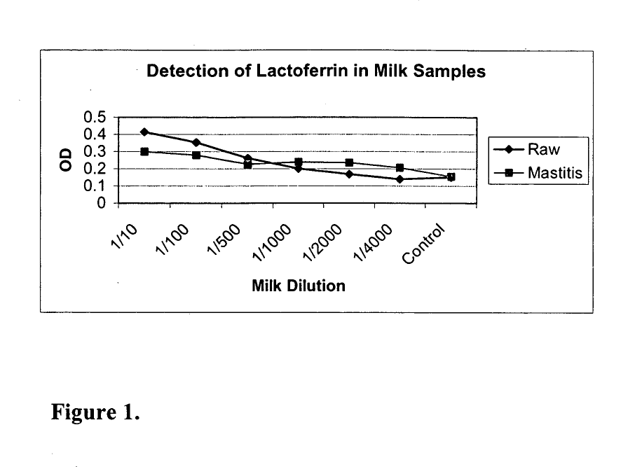

This example demonstrates that antibodies against lactoferrin can be used to detect the presence of mastitis in milk samples. FIG. 1 shows the results of a capture ELISA using an anti-lactoferrin monoclonal antibody against the indicated dilutions of raw milk. Antibodies were obtained from Bethyl Laboratories, RDI, Sigma-Aldrich.

Capture ELISA was performed using routine procedures as described below:
1. Added 50 μl of 1/1000 dilution of Anti-bovine lactoferrin antibody (1 mg/ml stock solution); sat at room temp for 30 min
2. Added 125 μl of 2% chicken serum (blocking agent); sat at room temp for 30 min
3. Added 50 μl of milk dilution(s) or lactoferrin; sat at room temp for 30 min.
4. Added 50 μl of biotinylated (labeled) anti-lactoferrin antibody (1 mg/ml stock solution); sat at room temp for 30 min
5. Added 50 μl of 1/500 EAP; sat at room temp for 30 min
6. Added 50 μl of ABTS; sat at room temp for 30 min
7. Read plate using 405 nm light when green color developed in step 6.

The plate was washed twice using 0.05% tween 20 and sodium PBS and once in sodium PBS. The plate was emptied of all liquid before the next step was added.

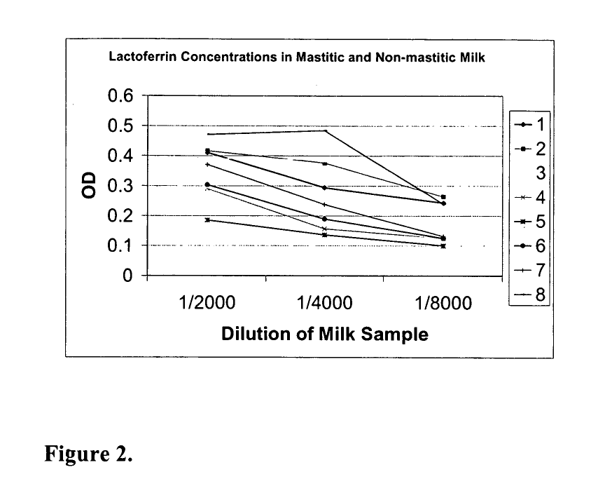

FIG. 2 depicts the difference in lactoferrin concentrations in various mastitis and non-mastitis samples, including mastitis infection with different organsisms, including Str. agalactiae, S. aureus, and Str. dysgalactiae. Raw milk was obtained from a local dairy processing plant, and mastitis milk was obtained from Washington State University Research Dairy. Sterile phosphate-buffered saline was used as a control.

These results clearly demonstrate that mastitis in milk samples can be detected using anti-lactoferrin antibodies and an ELISA format and, furthermore, establish that anti-lactoferrin antibodies can be used to detect mastitis in an immunological-based assay.

### Example 2

### Detection of Mastitis Using an Anti-Lactoferrin Antibody in an Immunochromatographic Assay

This example demonstrates that mastitis can be detected in a milk sample using a lateral flow immunological assay. Schematic diagrams of the principle of the lateral flow assay devised during this project and an exemplary lateral flow detection device are shown in FIGS. 3 and 4, respectively.

Lateral flow immunological assays were performed as depicted to optimize the relative concentrations of the capture antibody, biotin-labeled antibody and streptavidin-gold conjugate used for lateral flow detection. Various concentrations of the capture antibody, biotin-labeled antibody and streptavidin-gold conjugate were tested in order to optimize the assay. Capture and control lines were measured from the front end of the test device. Labeled antibodies were prepared using BiotinTag Micro-biotinylation Kit, Catalog B-Tag from Sigma Chemical Co., St Louis, Mo. This procedure was based on methods described by Jones, 1999, Millipore Corp., 2001, Chandler et al, 2000, Weiss, 1999 and Paek et al, 2000. A set of optimal concentrations of reagents for the detection of mastitis in a milk sample is shown in Table 1.

TABLE 1
Optimal concentrations of reagents for mastitis
detection by lateral flow assay.

| Material | Amount | Placement in Test |
|---|---|---|
| Goat-anti bovine antibody | 1 ul of | 0.7 cm from end of |
| (capture) | 1 mg/ml | test |
| Bovine lactoferrin | 1 ul of | 1.2 cm from end of |
| (control line) | 1 mg/ml | test |
| Goat anti-bovine lactoferrin | 24 ug | Conjugate Pad |
| antibody (biotinylated) | | |
| Streptavidin-goid conjugate | 10 ul | Conjugate Pad |
| | (OD 10) | |

Other components of the lateral flow device included nitrocellulose paper with mylar backing, a conjugate pad, a sample pad, an absorbent pad, a wick, and plastic housing. This assay used a nitrocellulose paper with a speed of 135 mm/4 min. The capture antibody and bovine lactoferrin were layered onto the nitrocellulose paper using a unique rubber stamp that enabled the material to be deposited in a uniform line.

The optimized lateral flow device was used to test for the presence of mastitis in sub-clinical mastitis (samples 1, 3 and 5) and non-mastitis milk samples (samples 2, 4, and 6). The results are depicted in Table 2. The times indicate when a distinct pink line (positive) developed.

TABLE 2
Results of lateral flow assay of mastitis and non-mastitis milk samples.

| | Capture | Control |
|---|---|---|
| 1 | 5.25 min | 1.75 min |
| 2 | None | 4.25 min |
| 3 | 1 min | 1 min |
| 4 | None | 1.25 min |
| 5 | 4.25 min | 2.75 min |
| 6 | None | 1.25 min |

Additional lateral flow assays were performed in duplicate on mastitic and non-mastitic milk samples using goat anti-bovine lactoferrin as depicted in FIGS. 3 and 4, according to the following protocol.
1) Obtain milk sample from teat.
2) Dilute milk 1/100 in Phosphate Buffered Saline (PBS); mix well.
3) Take lid off of test device and immerse dipstick ½ way into sample, dipstick will absorb approximately 1 ml of milk sample.
4) Replace lid and lay test device flat on a level surface.
5) Read test, two pink lines indicates a positive result and one pink line depicts a negative result.
6) DO NOT read test after 10 min, since false-positive tests may be observed.

The results of these assays are shown in Table 3. The milk samples used to run the test were obtained from the Washington State University Field Investigation Unit using cows with known mastitis infection. The infecting bacteria are listed with the corresponding sample number.

TABLE 3
Results of lateral flow assay of mastitis and non-mastitis milk samples.

| Sample # | Test1 | Test2 | Bacteria |
|---|---|---|---|
| 1 | Pos | Pos | S.uberis |
| 2 | Pos | Pos | S. aureus |
| 3 | Neg | Neg | Kiebsiella |
| 4 | Pos | Neg | Kiebsiella |
| 5 | Pos | Pos | Multiple |
| 6 | Pos | Pos | Corynebacterium |
| 7 | Neg | Neg | S. aureus |
| 8 | Neg | Neg | Coagulase Negative |
| | | | Staph |
| 9 | Neg | Neg | No Bacteria |
| 10 | Pos | Neg | Mycobacterium |
| 11 | Pos | Neg | Maycobacterium |
| 12 | Pos | Pos | S. aureus toxin |
| 13 | Pos | Pos | S. aureus toxin |
| 14 | Neg | Neg | No Bacteria |
| 15 | Pos | Pos | S.areus |
| 16 | Neg | Neg | No Bacteria |
| 17 | Pos | Neg | Str.dysgalactiae |
| 18 | Neg | Neg | No Bacteria |

The results of these experiments clearly demonstrate that the lateral flow assay can be distinguish between non-mastitic milk and sub-clinical mastitic milk.

All of the above U.S. patents, U.S. patent application publications, U.S. patent applications, foreign patents, foreign patent applications and non-patent publications referred to in this specification and/or listed in the Application Data Sheet, including but not limited to U.S. Pat. Nos. 5,622,871 and 6,352,862, and references and patents cited therein, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A device adapted for the detection of mastitis in an animal, comprising:
(a) a vessel to hold a milk sample;
(b) a first binding agent; and
(c) a second binding agent,
wherein the first binding agent binds a mastitis-associated molecule, and wherein the second binding agent comprises a detectable label.

2. (canceled)

3. The device of claim 1, wherein the second binding agent binds a mastitis-associated molecule.

4. The device of claim 3, wherein the first and/or second binding agent binds lactoferrin.

5. (canceled)

6. The method of claim 5, wherein the vessel is a solid support comprising a porous material.

7. (canceled)

8. The method of claim 5, wherein the vessel is a chromatography column.

9. The device of claim 1, wherein the binding agents are antibodies specific for lactoferrin.

10. (canceled)

11. The device of claim 5, wherein the solid support comprises the following components:
(a) a liquid sample application wick;
(b) a conjugate pad comprising the labeled binding agent;
(c) a base pad comprising the unlabeled binding agent;
(d) a sample pad comprising a blocking agent; and
(e) an absorbent pad.

12. The device of claim 5, further comprising a control binding agent that is immobilized in a second region of the solid support.

13. The device of claim 1, further comprising a detectable secondary agent that binds the labeled binding agent.

14. The device of claim 13, wherein the labeled binding agent is labeled with biotin, and the detectable secondary agent comprises strepavidin.

15. (canceled)

16. A kit adapted for the detection of mastitis in an animal, comprising:
(a) a first binding agent;
(b) a second binding agent; and
(c) instructions for the use of the kit,
wherein said first binding agent specifically bind a mastitis-associated molecule.

17. The kit of claim 16, further comprising a porous material to which a liquid sample may be applied.

18. (canceled)

19. The kit of claim 18, wherein the second binding agent comprises a label.

20. (canceled)

21. The kit of claim 19, further comprising a detectable secondary agent that binds the labeled binding agent.

22. The kit of claim 19, wherein the labeled binding agent is an antibody.

23-24. (canceled)

25. The kit of claim 19, wherein the labeled binding agent is labeled with biotin and the secondary agent comprises streptavidin.

26-27. (canceled)

28. A method adapted for detecting mastitis in an animal, comprising:
(a) incubating a sample of milk from an animal with a labeled binding agent that binds a mastitis-associated molecule; and
(b) detecting binding agent bound to the sample, thereby detecting mastitis in the animal.

29-30. (canceled)

31. The method of claim 28, wherein the binding agent is a monoclonal antibody specific for lactoferrin.

32-36. (canceled)

37. An analytical test device for detecting the presence of mastitis in an animal, comprising:
(a) a hollow casing having a liquid sample application aperture and a means permitting observation of a test result; and
(b) a test strip comprising a dry porous material contained within said hollow casing,
said test strip communicating directly or indirectly with the exterior of said hollow casing through said liquid sample application aperture to receive applied liquid sample, said test strip having a capture line observable via said means permitting observation, said test strip, in the dry unused state, containing a labeled agent capable of specifically binding lactoferrin to form a first complex of said labeled agent and said bacteria, wherein said labeled agent is dry on said test strip prior to use and is released and mobile upon application of said applied liquid sample, and
said test strip containing in said capture line a means for specifically binding said first complex, said means for binding being immobilized in said capture line; wherein said binding means binds said first complex to form a second complex, said second complex being observable via said means permitting observation, thereby to indicate the presence of mastitis in the animal.

38. (canceled)

39. The device of claim 3637, further comprising a control line downstream from said capture line in said dry porous carrier to indicate that said liquid sample is conveyed beyond said capture line, and a control line observation aperture in said casing, said control line also being observable from outside said hollow casing through said control line observation aperture.

40. The device of claim 39, wherein said control line contains a means for binding said labeled agent and wherein said means is immobilized in said control line.

41-44. (canceled)
